(19) United States
(12) Patent Application Publication
(10) Pub. No.: US 2022/0365642 A1
(43) Pub. Date: Nov. 17, 2022

(54) METABOLIC NETWORK EXPLORER

(71) Applicant: SRI International, Menlo Park, CA (US)

(72) Inventors: Peter D. Karp, San Mateo, CA (US); Suzanne M. Paley, Redwood City, CA (US)

(21) Appl. No.: 17/660,158

(22) Filed: Apr. 21, 2022

Related U.S. Application Data
(60) Provisional application No. 63/189,373, filed on May 17, 2021.

(51) Int. Cl. G06F 3/0484; G16B 5/00; G16B 40/20; G06F 3/0482

(52) U.S. Cl. CPC G06F 3/0484; G16B 5/00; G16B 40/20; G06F 3/0482

(57) ABSTRACT

This disclosure describes techniques for generating a visual path through a metabolic network, techniques include receiving reaction data for a starting metabolite, the reaction data comprising one or more potential reaction steps of the starting metabolite with one or more neighbor metabolites, generating from the one or more neighbor metabolites, a precursor metabolite list of precursor metabolites and a successor metabolite list of successor metabolites based on the reaction data, generating a user interface comprising the visual path that includes interactive visual data, the interactive visual data indicating each metabolite from the precursor metabolite list and the successor metabolite list, wherein each metabolite from the precursor metabolite list and the successor metabolite list is selectable and indicates a corresponding potential reaction step of the one or more potential reaction steps of the starting metabolite, and outputting the user interface for display at a display device.

This application claims the benefit of U.S. Provisional Patent Application No. 63/189,373, filed on May 17, 2021, and entitled “METABOLIC NETWORK EXPLORER,” which is incorporated herein by reference in its entirety.

### GOVERNMENT INTEREST

This invention was made with Government support under grant number 5R01GM08074 awarded by the National Institutes of Health. The Government has certain rights in this invention.

### TECHNICAL FIELD

This disclosure relates to metabolic pathways and, in particular, to a computing system for dynamically visualizing metabolic pathways.

### BACKGROUND

A metabolic network describes the relationships between small biomolecules (metabolites) and the enzymes (proteins) that interact with them to catalyze a biochemical reaction. As such, the metabolic network is a complete set of metabolic and physical processes that determine the physiological and biochemical properties of a cell. These networks comprise the chemical reactions of metabolism, the metabolic pathways, as well as regulatory interactions that guide these reactions. A metabolic pathway is a series of successive reactions that changes one or more educts into one or more products. A particular metabolite may have a multitude of initial pathways, where each pathway also may include a multitude of pathways in a branching effect. A typical metabolite network mapping is a two or three dimensional map that illustrates a multitude of branching pathways in the metabolic network and its sub-networks.

### SUMMARY

In general, techniques are described for generating a user interface that includes an interactive metabolic network map. For example, a metabolic network exploration system may be configured to generate and navigate a complex metabolic network map generated around a starting metabolite. The techniques may enable an easy and interactive navigation of a portion of a metabolic network. In some examples, unlike other more general metabolic network visualization tools, no prior preselection or preloading of a sub-network is required beyond an initial selection of the starting metabolite and database of interest. Because the techniques may limit the database access and subsequent reaction path generation to a single linear path at a time, the techniques may allow for a compact, easy-to-read, aesthetically appealing, dynamic, and interactive user interface to display the metabolic map of the metabolic network of interest, which may increase research efficiency into the metabolic network of interest. The user interface may provide selectable predecessor and successor metabolite lists for every metabolite in the selected reaction path, thus making it easy to identify and efficiently explore alternate paths. The user interface may also provide ready access to detailed information pages for each reaction, pathways and enzymes associated with the current path, and fills an important gap in the set of metabolic network visualization tools.

In one example, the disclosure describes a method for generating a visual path through a metabolic network. The method includes receiving, by a computing system, first reaction data for a starting metabolite, the first reaction data comprising one or more potential reaction steps of the starting metabolite with one or more first neighbor metabolites; generating, by the computing system, from the one or more first neighbor metabolites, a first precursor metabolite list of precursor metabolites and a first successor metabolite list of successor metabolites based on the first reaction data; generating, by the computing system, a user interface comprising the visual path that includes interactive visual data, the interactive visual data indicating each metabolite from the first precursor metabolite list and the first successor metabolite list, wherein each metabolite from the first precursor metabolite list and the first successor metabolite list is selectable and indicates a corresponding potential reaction step of the one or more potential reaction steps of the starting metabolite; and outputting, by the computing system, the user interface for display at a display device. In another example, the disclosure describes a metabolic network exploration system comprising a storage device and processing circuitry having access to the storage device. The processing circuitry configured to receive first reaction data for a starting metabolite, the first reaction data comprising one or more potential reaction steps of the starting metabolite with one or more first neighbor metabolites; generate from the one or more first neighbor metabolites, a first precursor metabolite list of precursor metabolites and a first successor metabolite list of successor metabolites based on the first reaction data; generate a user interface comprising the visual path that includes interactive visual data, the interactive visual data indicating each metabolite from the first precursor metabolite list and the first successor metabolite list, wherein each metabolite from the first precursor metabolite list and the first successor metabolite list is selectable and indicates a corresponding potential reaction step of the one or more potential reaction steps of the starting metabolite; and output the user interface for display at a display device.

In another example, the disclosure describes a non-transitory, computer-readable medium comprising instructions for causing one or more programmable processors to: receive first reaction data for a starting metabolite, the first reaction data comprising one or more potential reaction steps of the starting metabolite with one or more first neighbor metabolites; generate from the one or more first neighbor metabolites, a first precursor metabolite list of precursor metabolites and a first successor metabolite list of successor metabolites based on the first reaction data; generate a user interface comprising the visual path that includes interactive visual data, the interactive visual data indicating each metabolite from the first precursor metabolite list and the first successor metabolite list, wherein each metabolite from the first precursor metabolite list and the first successor metabolite list is selectable and indicates a corresponding potential reaction step of the one or more potential reaction steps of the starting metabolite; and output the user interface for display at a display device.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a block diagram that illustrates a computing system for generating a visual path through a metabolic network, in accordance with techniques of the disclosure.

FIG. 2 is a block diagram illustrating a computing system for generating a visual path through a metabolic network, in accordance with techniques of the disclosure.

FIGS. 3A-3F illustrate user interfaces for displaying and interfacing with a metabolic network, in accordance with techniques of the disclosure.

FIG. 4 is a flow diagram illustrating an example operation of a computer system for generating a visual path through a metabolic network, in accordance with techniques of the disclosure.

Like reference characters refer to like elements throughout the figures and description.

### DETAILED DESCRIPTION

This disclosure describes inventive concepts with reference to specific examples. However, the intent is to cover all modifications, equivalents, and alternatives of the inventive concepts that are consistent with this disclosure. It will be apparent, however, to one of ordinary skill in the art that the present approach can be practiced without these specific details. Thus, the specific details set forth are merely exemplary, and is not intended to limit what is presently disclosed. The features implemented in one embodiment may be implemented in another embodiment where logically possible. The specific details can be varied from and still be contemplated to be within the spirit and scope of what is being disclosed.

While modern science has come a long way in understanding how the human body works, researchers today are still uncovering new information around how different metabolic mechanisms impact organisms and the environment. There is a great need for scientist to understand how the metabolic network of an organism can biochemically interconvert different chemical compounds. The disclosed system may provide route searching capabilities that automatically search for connecting metabolic routes and may graphically display portions of metabolic networks that include the metabolic routes.

Metabolic network maps are large and complex graphs that can be daunting to comprehend and difficult to navigate to and around specific metabolites and their neighboring metabolic paths. Past research has provided multiple modes for exploring metabolic networks. Highly localized modes include viewing single reactions, single metabolites, and the reactions they participate in, and viewing predefined metabolic pathways, either singly or in connected groupings. These approaches lack larger network context. Global modes of exploration include visualization of entire metabolic networks, or more precisely, of all metabolites combined with a limited set of reaction edges, for including all reactions for each pathway typically produces an incomprehensible tangle.

The disclosed metabolic network exploration system fills a gap in the existing modes of exploration that enables a user to begin at a starting metabolite and interactively explore the metabolic neighborhood around that starting metabolite by moving forward into successor metabolites and backward into precursor metabolites in the network to build up a linear visual reaction path of arbitrary length. Thus, the metabolic network exploration system enables automated exploration and navigation through connected sets of reactions without the use of predefined pathways, and further provides a multitude of options to display complex metabolite reaction data, including formulas, enzyme and gene data, metabolite structures, Enzyme Commission (EC) number, and shortcuts to prior displayed metabolic pathways.

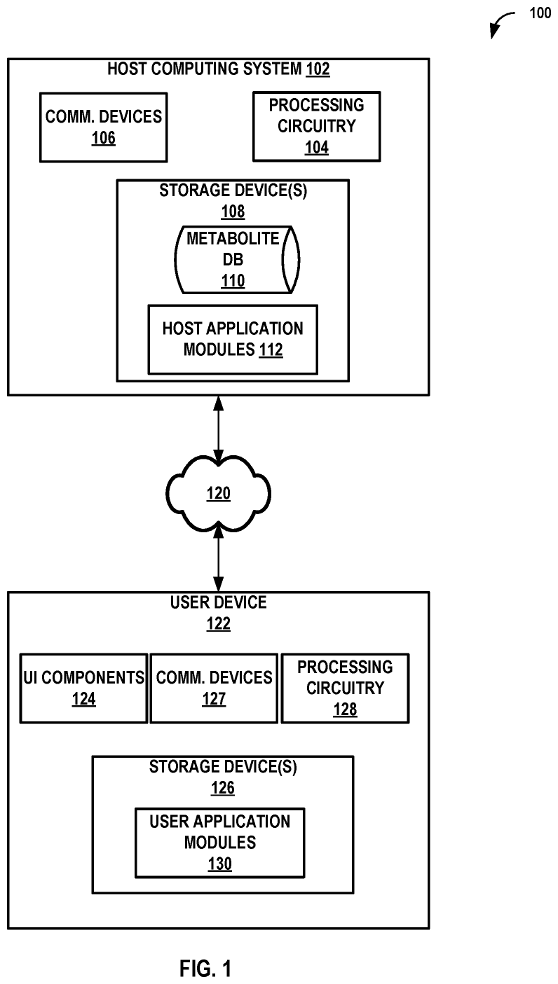

FIG. 1 is a block diagram that illustrates a metabolic network exploration system 100 for generating a visual path through a metabolic network, in accordance with techniques of the disclosure. Metabolic network exploration system 100 is a computing system that includes host computing system 102 connected to user device 122. In one example, host computing system 102 and user device 122 are connected via a network 120. Although primarily herein described as being implemented using a distributed system that includes a host computing system and one or more user devices that access the host computing system, metabolic network exploration system 100 may be implemented using a centralized system in which aspects of host computing system 102 and user device 122 are combined into one computing device.

Host computing system 102 may be implemented as any suitable computing system, such as one or more server computers, workstations, laptops, mainframes, appliances, cloud computing systems, smart phones, tablet computers, and/or other computing systems that may be capable of performing operations and/or functions described in accordance with one or more aspects of the present disclosure. In some examples, host computing system 102 may represent a cloud computing system, server farm, and/or server cluster (or portion thereof) that provides services to client devices and other devices or systems, such as user device 122. In other examples, host computing system 102 may represent or be implemented through one or more virtualized compute instances (e.g., virtual machines, containers, etc.) of a data center, cloud computing system, server farm, and/or server cluster. Components of host computing system 102 may distributed among one or more compute nodes, storage nodes, application nodes, web servers, or other computing devices.

Host computing system 102 includes processing circuitry 104, one or more communication devices 106, and one or more storage devices 108. Processing circuitry 104, in one example, is configured to implement functionality and/or process instructions for execution within host computing system 102. One or more storage devices 108 may be configured to store information, such as metabolite database 110 and executable code associated with host application modules 112 within host computing system 102 during operation. Host computing system 102, in one example, uses communication devices 106 to communicate with external devices via one or more networks, such as network 120.

Network 120 is a communications network that connects host computing system 102 and user device 122). The network 120 may represent or include one or more of an optical network, a cellular network, the Internet, a Local Area Network (LAN), Wide Area Network (WAN), satellite link, fiber network, cable network, or a combination of these and/or others.

In one example, metabolic network exploration system 100 for mapping a metabolic network in accordance with the techniques of the disclosure may be implemented as a hosted website by host computing system 102 communicating over network 120. The host computing system 102 may include host application modules 112, which may include a webserver application or other application for communicating with user device 122. In one example, storage devices 108 may include host application modules 112 and metabolite database 110 and may be accessed and used by processing circuitry 104 to receive input (e.g., a starting metabolite) from user device 122 to generate a metabolic network map for display on user device 122.

The metabolite database 110, for example, may include a collection of Pathway/Genome Databases (PGDBs), which describes the genes, metabolism, and other functions of certain organisms or collections of organisms, such as EcoCyc, a database for the single organism E. coli K-12 MG1655, and MetaCyc, a database of metabolic reactions and enzymes from all branches of life. In one example, the metabolite database 110 is hosted remotely by a server computer system (e.g., metabolite database 110 of host computing system 102) and accessed by a client computer system (e.g., user device 122). In other examples, a user application (e.g., user application modules 130) may be hosted locally, thus allowing a user to build a custom PGDB for any organism of interest from its annotated genome and use the hosted applications to interactively explore its predicted metabolic network.

User device 122 may be operated by a user. User device 122 may be implemented as any suitable client computing system, such as a mobile, non-mobile, wearable, and/or non-wearable computing device. User device 122 may represent a smart phone, a tablet computer, a computerized watch, a personal digital assistant, a virtual assistant, a gaming system, a media player, an e-book reader, a television or television platform, a laptop or notebook computer, a desktop computer, a camera, or any other type of wearable, non-wearable, mobile, or non-mobile computing device that may perform operations in accordance with one or more aspects of the present disclosure.

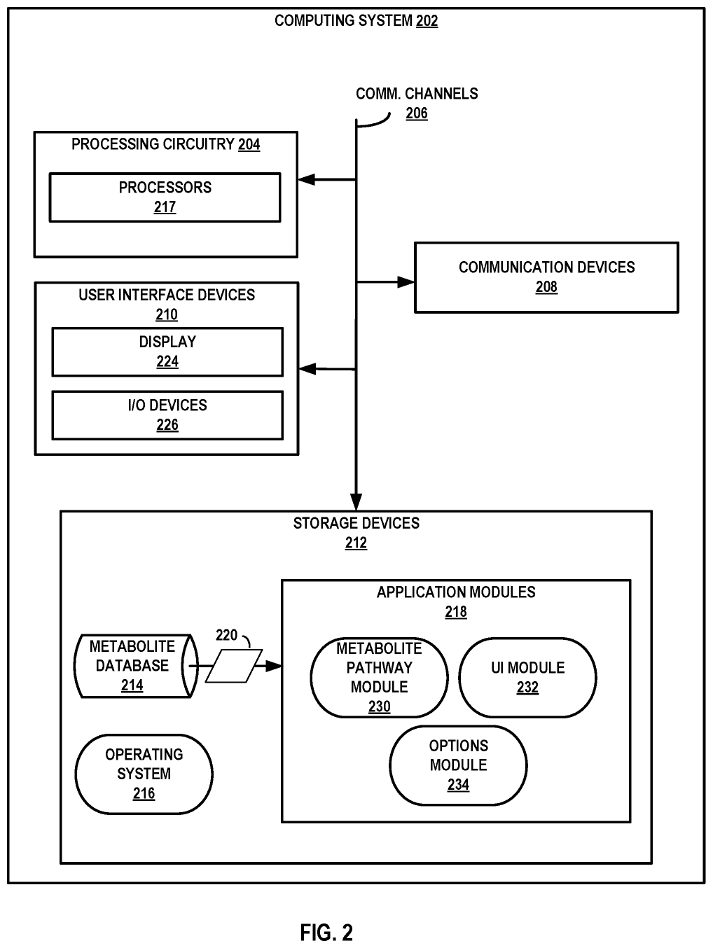

User device 122 may include user interface components 124, storage devices 126, communication devices 127, processing circuitry 128, and user application modules 130. Processing circuitry 128, in one example, is configured to implement functionality and/or process instructions for execution within user device 122. One or more storage devices 126 may be configured to store information and executable code associated with user application modules 130 within user device 122 during operation. User device 122, in one example, uses communication devices 127 to communicate with external devices via one or more networks, such as network 120. In one example, user application modules 130 include a web browser or other applications executable by processing circuity 128 to communicate with host computing system 102 over network 120. Example user interface components are described below with respect to user interface devices 210 of computing system 202 of FIG. 2.

A precursor metabolite is any metabolite such that a reaction exists with the precursor metabolite as a reactant and the starting metabolite as a product. A successor metabolite is any successor metabolite such that a reaction exists with the starter metabolite as a reactant and successor metabolite as a product. Some reactions are reversible or of unspecified directionality. In such examples, a metabolite could be either a predecessor or a successor, so is included in both categories and thus both precursor metabolite list and successor metabolite list.

In accordance with techniques of this disclosure, metabolic network exploration system 100 generates a user interface that includes an interactive metabolic network map of a metabolic network. For example, metabolic network exploration system 100 obtains an indication of a starting metabolite from a user. User device 122, for example, may receive user input indicating the starting metabolite and send the indication to host computing system 102. Host application modules 112 process the indication of the starting metabolite to obtain, from metabolite database 110, first reaction data for the starting metabolite. The first reaction data includes one or more potential reaction steps of the starting metabolite with one or more first neighbor metabolites. Host application modules 112 may generate, from the one or more first neighbor metabolites, a first precursor metabolite list of precursor metabolites and a first successor metabolite list of successor metabolites based on the first reaction data. Host computing system 102 may send the first precursor metabolite list of precursor metabolites and the first successor metabolite list of successor metabolites to user device 122. User application modules 130 may process the first precursor metabolite list of precursor metabolites and the first successor metabolite list of successor metabolites to generate a user interface comprising a visual path through a metabolic network. The visual path includes interactive visual data indicating each metabolite from the first precursor metabolite list and the first successor metabolite list. Each metabolite from the first precursor metabolite list and the first successor metabolite list may be selectable (e.g., by a user providing user input via user interface components 124) and indicates a corresponding potential reaction pathway of the one or more reaction pathways of the starting metabolite. User device 122 outputs the user interface for display at a display device.

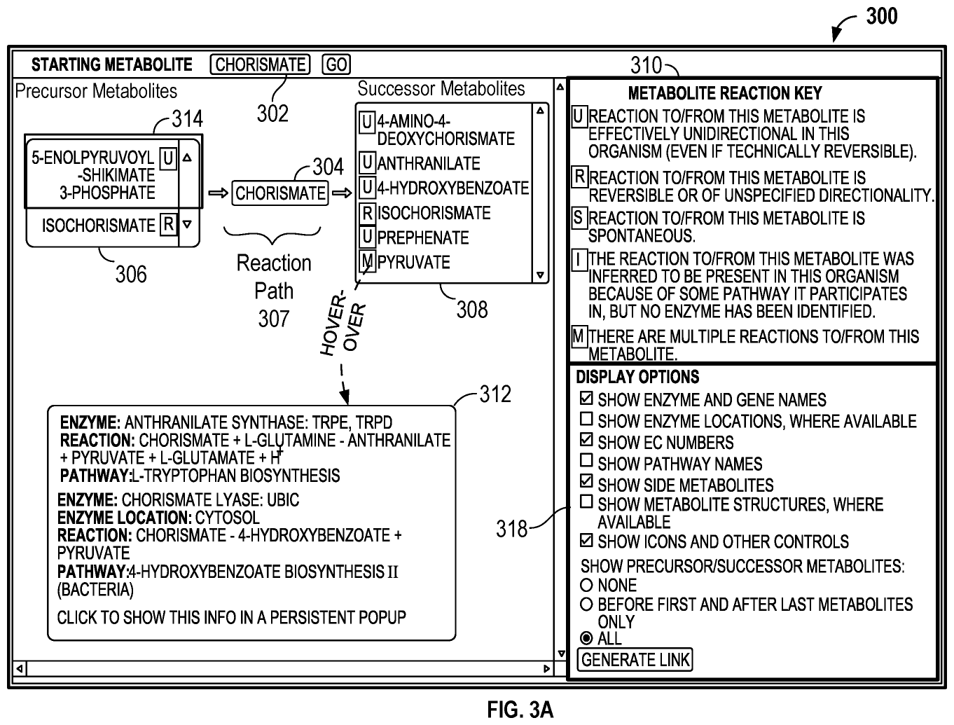

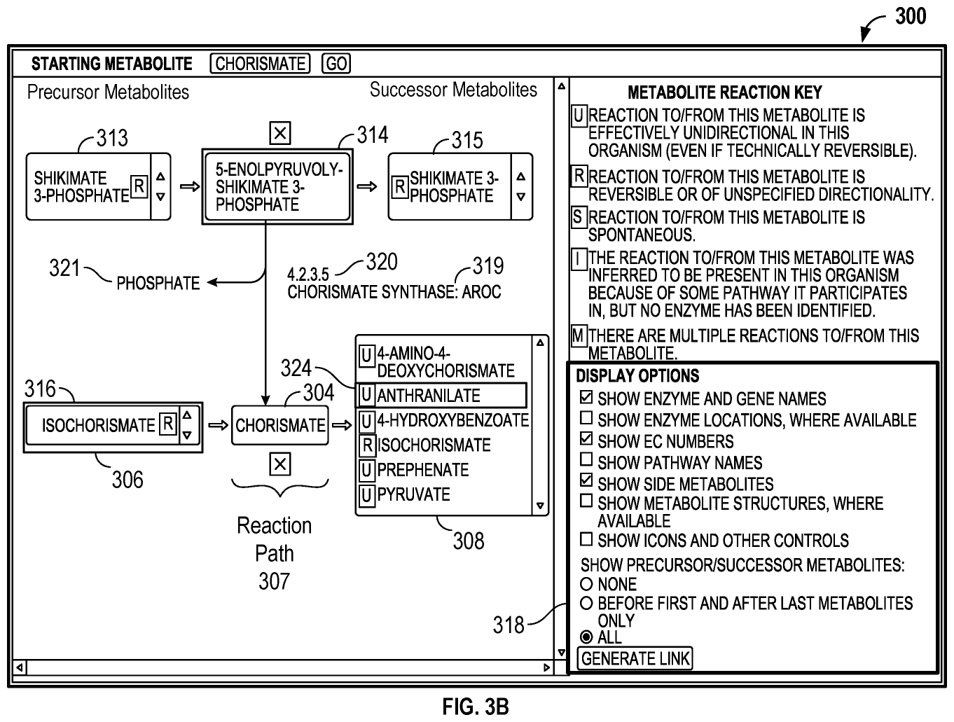

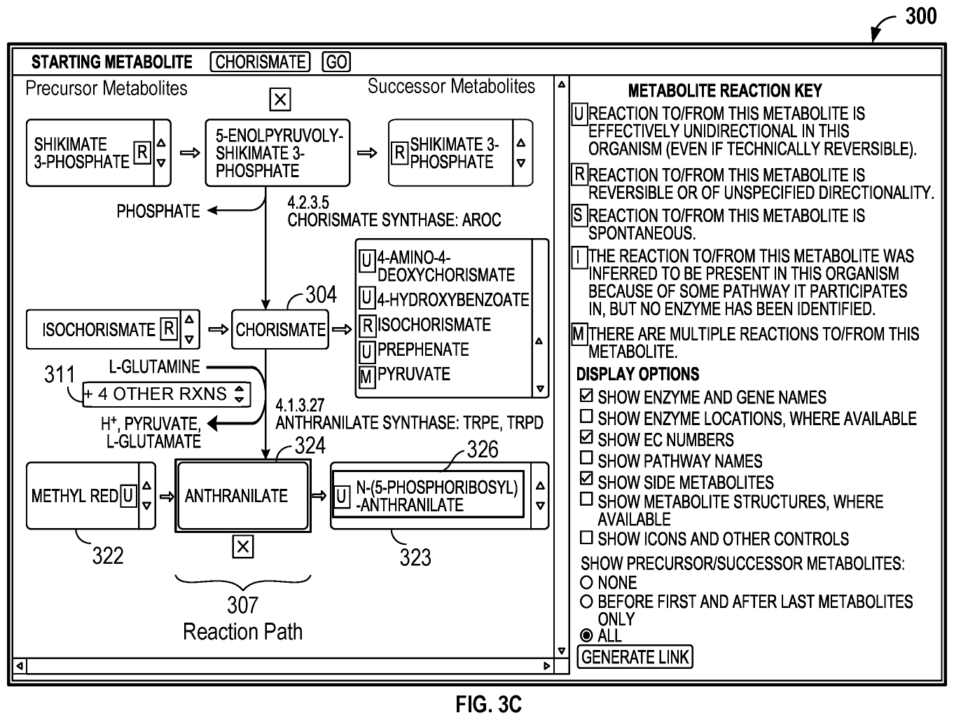

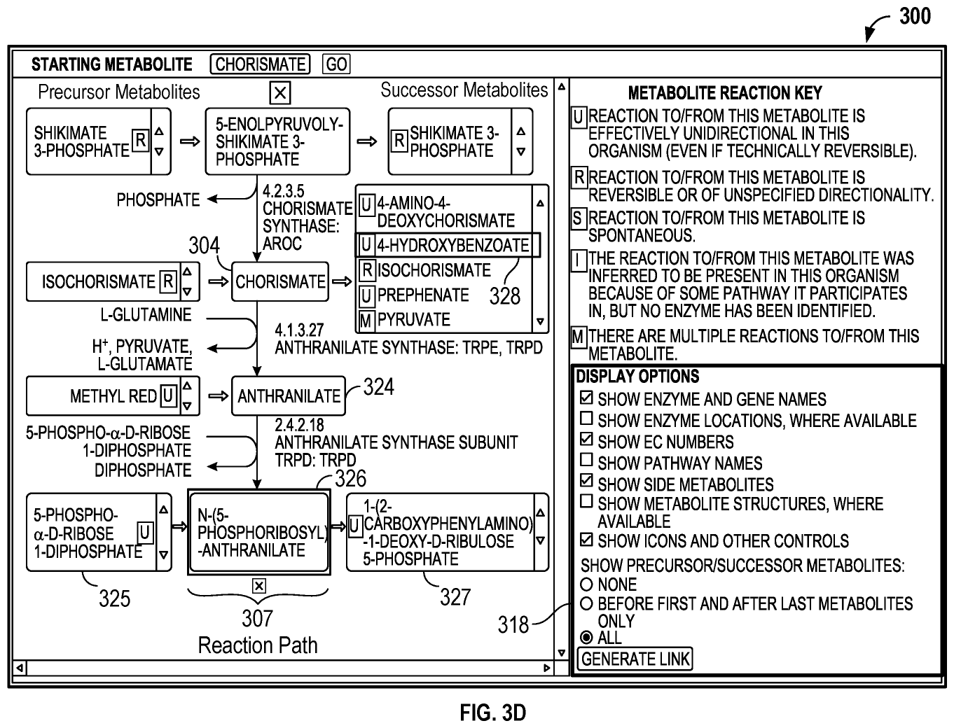

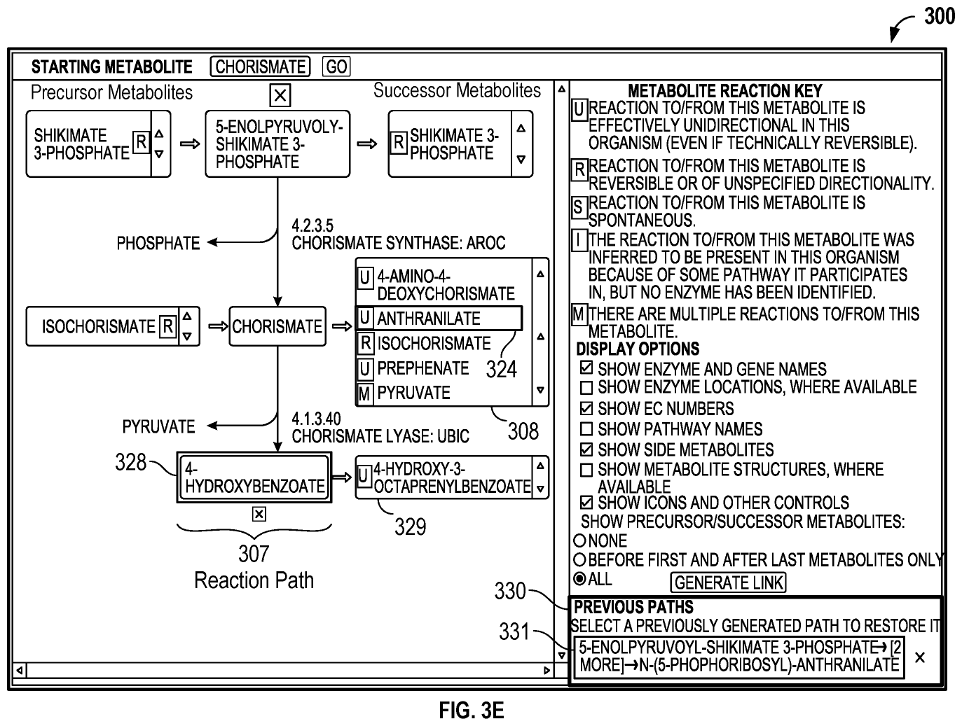

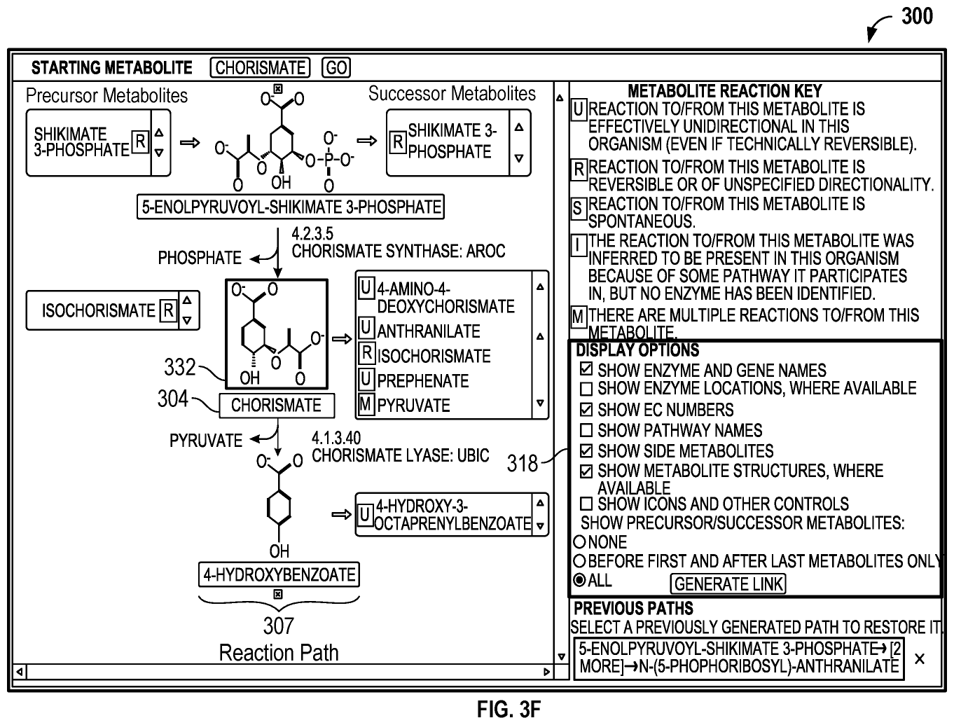

The techniques may enable an easy and interactive navigation of a portion of a metabolic network. In some examples, unlike other more general metabolic network visualization tools, no prior preselection or preloading of a sub-network is required beyond an initial selection of the starting metabolite and metabolic database of interest (e.g., metabolic database 110). Because the techniques may reduce metabolic database access and subsequent reaction path generation to a single linear path at a time, the techniques may allow for a compact, easy-to-read, aesthetically appealing, dynamic, and interactive user interface to display the portion of the metabolic map of interest (see e.g., reaction path 307 of FIGS. 3A-3F), which may increase research efficiency into the metabolic network of interest. The user interface may provide selectable predecessor and successor metabolite lists for every metabolite in the selected reaction path, thus making it easy to identify and efficiently explore alternate paths. The user interface may also provide ready access to detailed information for each reaction, pathways and enzymes associated with the current visual path, and fills an important gap in the set of metabolic network visualization tools. In one example, the metabolic neighborhood of the starting metabolite is a JavaScript object that includes a list of possible precursor and successor metabolites based on the set of reactions in a metabolic database (e.g., metabolic database 110). A small number of ubiquitous inorganic molecules, such as water, H+, and phosphate, may be omitted from the precursor list and the successor list of predecessor and successor metabolites. In various examples, operations attributed above to user application modules 130 may be performed by host application modules 112, and operations attributed above to host application modules 112 may be performed by user application modules 130.

FIG. 2 is a block diagram illustrating a computing system 202 for generating a visual path through a metabolic network, in accordance with techniques of the disclosure. FIGS. 3A-3F illustrate a user interface 300 for generating and interfacing with a metabolic network in accordance with techniques of the disclosure. Computing system 202 may generate, for output at display device 224, information to a user in the form of a user interface, which may be associated with functionality provided by computing system 202. Such user interfaces may be associated with computing platforms, operating systems, applications, and/or services executing at or accessible from computing system 202. For example, display device 224 may present one or more user interfaces which are graphical user interfaces of application(s) executing at computing system 202, the user interfaces including various graphical elements displayed at various locations of display device 224, such as text boxes, check boxes, ring menus, active inputs, etc. Although illustrated as part of computing system 202, display device 224 may be a separate device.

In various examples, components and modules illustrated in computing system 202 of FIG. 2 for generating a user interface depicting a visual path through a metabolic network, as illustrated in FIGS. 3A-3F, may be implemented, in whole or in part, by host computing system 102 and/or user device 122 of FIG. 1. For example, a network environment host computing system 102 (server) may provide some or most of the functionality, compute and storage (e.g., via processing circuitry 104, host application modules 112 and metabolite database 110). Alternatively, some of the functionality, compute, and storage (e.g., via processing circuitry 128, user application modules 130 and storage devices 126) may be provided at least in part by a stand-alone application on user device 122 with some to no communication with the host computing system 102. For example, the metabolite database may be hosted on the host computing system 102 or entirely on the user device 122, or on one or more other computing devices. Computing system 202 may be an example of or implement any metabolic network exploration system described in this disclosure. For simplicity, the example of FIG. 2 illustrates techniques of the disclosure in a single computer system but is not limited to such.

In one example, computing system 202 includes processing circuitry 204, communication channels 206, communication devices 208, user interface devices 210, and storage devices 212. Storage devices 212, in one example, further includes a metabolite database 214, an operating system 216, and application modules 218. In one example, operating system 216 and application modules 218 are executable by processing circuitry 204 to generate pathways around a starting metabolite in a metabolic network map in accordance with the techniques of the disclosure.

Storage devices 212, in some examples, may include a computer-readable storage medium and may further include one or more memory devices such as volatile and non-volatile memories. For example, random access memories (RAM), dynamic random access memories (DRAM), static random access memories (SRAM), magnetic hard discs, optical discs, floppy disks, Flash memories, electrically programmable memories (EPROM) or electrically erasable and programmable (EEPROM) memories and other forms of memories known in the art.

Processing circuitry 204, in one example, is configured to implement functionality and/or process instructions for execution within computing system 202. Examples of processing circuitry 204 may include any one or more of a microprocessor, a controller, a digital signal processor (DSP), an application specific integrated circuit (ASIC), a field-programmable gate array (FPGA), or equivalent discrete or integrated logic circuitry.

In one example, processing circuitry 204 may process application and operating system instructions and access data stored in storage devices 212, which include application modules 218, operating system 216 and metabolite database 214. Operating system 216, in some examples, controls the operation of components of computing system 202, such as managing communications between communication devices 208, storage devices 212, communication channels 206, and user interface devices 210.

Computing system 202, in one example, utilizes communication devices 208 to communicate with external devices via one or more networks, such as one or more wired/wireless/mobile networks. Communication devices 208 may include a network interface card, such as an Ethernet card, an optical transceiver, a radio frequency transceiver, or any other type of device that can send and receive information. Other examples of such network interfaces may include 3G, 4G, 5G, Bluetooth™, IEEE 802.15.4 (e.g., ZigBee™) and Wi-Fi™ radios. In some examples, computing system 202 uses communication devices 208 to communicate with one or more external devices, such as other user systems, host systems and data stores (e.g., databases).

User interface devices 210 of computing system 202 may include components for input and output for computing system 202. For example, one or more input/output (I/O) devices 226 and display device 224 of user interface devices 210 may receive inputs and generate outputs. Examples of inputs are tactile, audio, kinetic, and optical input, to name a few examples. I/O devices 226 may include a touchscreen integrated into display device 224, a touch pad, a mouse, a keyboard, a voice responsive system, a video camera, buttons, a control pad, a microphone, or any other type of device for detecting input from a human or machine. For example, display device 224 of user interface devices 210 may function as an input device using a resistive touchscreen, a surface acoustic wave touchscreen, a capacitive touchscreen, a projective capacitance touchscreen, a pressure sensitive screen, an acoustic pulse recognition touchscreen, or another presence-sensitive screen technology. The display device 224 may receive tactile user input by detecting one or more taps and/or gestures from a user of computing system 202, such as a user touching or pointing to one or more locations of display device 224 with a finger or a stylus pen. Further, display device 224 may function as an output device using any one or more of a liquid crystal display (LCD), dot matrix display, light emitting diode (LED) display, miniLED, microLED, organic light-emitting diode (OLED) display, e-ink, or similar monochrome or color display capable of outputting visible information to the user of computing system 202. Output device examples, in addition to display device 224, may include a sound card, a video graphics adapter card, a speaker, or any other type of device for generating output to a human or machine.

Application modules 218 may interpret input data detected by user interface devices 210. Application modules 218 may provide the input data to one or more associated platforms, operating systems, applications, and/or services executed by or for computing system 202 to cause computing system 202 to perform a function in accordance with one or more aspects of the present disclosure. In addition, one or more application modules 218 may also receive data and instructions from one or more associated platforms, operating systems, applications, and/or services executing at computing system 202 to further perform some or all of the functions described herein.

Application modules 218 may include metabolite pathway module 230, user interface (UI) module 232, and options module 234 and are executable from storage devices 212 by processing circuitry 204. As discussed above, for simplicity the components have been aggregated into computing system 202. In some examples, the functionality of metabolite database 214, metabolite pathway module 230, user interface (UI) module 232, and options module 234 may be split in a server-client based system, such as among host application modules 112 and metabolite database 110 of host computing system 102 and user application modules 130 of user device 122.

In one example, metabolite pathway module 230, UI module 232, and options module 234 are implemented in part as a browser application where UI module 232 is configured to generate a user interface, such as illustrated in FIGS. 3A-3F, using one or more of HyperText Markup Language (HTML), JavaScript and Cascading Style Sheets (CSS). The metabolite pathway module 230 may receive input from I/O devices 226 that includes input 302 of FIG. 3A used to receive a starting metabolite (e.g., starting metabolite 304 of user interface 300) to generate a metabolic reaction path (e.g., reaction path 307) that includes the immediate metabolic neighborhood around the starting metabolite. The metabolite pathway module 230 may query metabolite database 214 with the starting or selected metabolite and in response receive (metabolite) reaction data 220 from metabolite database 214.

The reaction data 220 received from the query of metabolite database 214 may indicate potential reaction steps from the starting or selected metabolite to neighbor metabolites and from neighbor metabolites to the starting or selected metabolite, and metabolite pathway module 230 may generate from the reaction data 220 a precursor list of selectable precursor metabolites and a successor list of selectable successor metabolites to include in the visual path through the metabolic network. The provided interactive user interface enables accelerated research capabilities by automated exploration and navigation through connected sets of reactions without the use of predefined pathways and provides a multitude of options to display complex metabolite reaction data quickly and efficiently.

In one example, the UI module 232 may generate user interface 300 to display on display device 224 a reaction path 307 that includes the starting metabolite 304 and reaction data including interactive visual data corresponding to precursor metabolite list 306 and successor metabolite list 308 in the neighborhood of metabolites that potentially react with starting metabolite 304. In one example, a web browser, or other suitable application, displays the user interface 300 of reaction path 307 that includes the starting metabolite 304. The user interface 300 may also include potential reaction steps from the starting metabolite in precursor metabolite list 306 in the column to the left of the starting metabolite 304 and the successor metabolite list 308 in the column to the right of the starting metabolite 304, as shown in FIGS. 3A-3F. The UI module 232 may also provide additional information to the user interface 300, such as metabolite reaction key 310 and display options 318. Metabolite reaction key 310 provides a key for the letter icons located next to each metabolite in precursor metabolite list 306 and successor metabolite list 308. Each letter indicates whether the reaction to and/or from starting metabolite 304 is unidirectional, potentially bidirectional, spontaneous, or if there are multiple reactions.

In one example, when a user hovers over any precursor or successor metabolite in user interface 300, UI module 232 retrieves information about the linking reaction(s) to the starting metabolite 304 from storage devices 212 (e.g., metabolite database 214) and updates the user interface 300 to include a pop-up text box 312 of reaction data. A user may select any metabolite from either precursor metabolite list 306 or successor metabolite list 308 to cause metabolite pathway module 230 and UI module 232 to update the user interface 300 to add that metabolite path to the displayed reaction path 307. To select a metabolite, the user may use an input device to provide a user input indicating selection of a corresponding letter icon for or an area of user interface 300 associated with the metabolite. In one example, the update to the user interface 300 may include the added metabolite, a visual path indicated by an arrow from the selected metabolite to the starting metabolite, a precursor metabolite list, and a successor metabolite list. If the metabolite added is a precursor to the starting metabolite in the visual path or a successor metabolite to the last metabolite in the visual path, the visual path indicated by an arrow is extended in that direction (up or down) on display device 224. For example, a user selects precursor metabolite 314 from precursor metabolite list 306 as shown in FIG. 3A. Turning to FIG. 3B, reaction path 307 is updated and a visual path extends upward from starting metabolite 304 and is an indicator to show precursor metabolite 314 connected to starting metabolite 304. In this example, because the selected metabolite is a precursor to the starting metabolite 304, the visual path of reaction path 307 is indicated by an arrow down from the precursor metabolite 314 to the starting metabolite 304 indicating the direction of the reaction step. In addition to including precursor metabolite list 306 and successor metabolite list 308, the updated user interface 300 further includes selectable precursor metabolite list 313 and successor metabolite list 315 associated with precursor metabolite 314.

User interface 300 includes display options 318 in the form of checkboxes. Display options 318 may include, but are not limited to, listing of enzymes and genes that catalyze the reaction, side metabolites, enzyme commission (EC) number, pathway names, or catalyst locations for each reaction pathway, metabolite structures, display control elements, and so on (see e.g., display options 318 of FIG. 3B). In one example, the boxes checked in display options 318 provide an indication to UI module 232 and options module 234 to retrieve and update the user interface 300 to display data from the metabolite database 214 corresponding to the checked boxes. For example, FIG. 3B illustrates, in proximity to the reaction path indicator, enzyme and gene name 319, EC number 320 and side metabolite 321, as indicated for display by the checked boxes of display options 318.

FIG. 3C illustrates automatically updating the user interface 300 from FIG. 2B when successor metabolite 324 associated with starting metabolite 304 is selected from successor metabolite list 315. In one example, the metabolite pathway module 230 queries metabolite database 214 with successor metabolite 324 and, in response, receives reaction data 220 from metabolite database 214 for successor metabolite 324. Reaction data 220 includes potential reaction steps from the successor metabolite 324 to neighbor metabolites. Metabolite pathway module 230 generates from reaction data 220 a precursor list of selectable precursor metabolites and a successor list of selectable successor metabolites. The UI module 232 updates user interface 300 to display on display device 224 updated reaction path 307 that includes the starting metabolite 304 and successor metabolite 324 and their respective precursor list of selectable precursor metabolites and a successor list of selectable successor metabolites. Thus, an extension of reaction path 307 downward from starting metabolite 304 is shown when successor metabolite 324 is selected from successor metabolite list 308 in FIG. 3B. The indicator corresponding to reaction path 307 is updated to include the reaction of adding successor metabolite 324 to starting metabolite 304. In this example, because the selected metabolite is a successor to the starting metabolite 304, the visual path of reaction path 307 is indicated by an arrow down from the starting metabolite 304 to successor metabolite 324 that indicates the direction of the reaction step. The updated user interface 300 includes prior selected metabolites in reaction path 307 and their respective selectable metabolite lists, and selectable precursor metabolite list 322 and successor metabolite list 323 associated with successor metabolite 324. The updated reaction path 307 further shows successor metabolite 324's selectable precursor metabolite list 322 and successor metabolite list 323.

Different reactions connecting two metabolites may have different side compounds and different other information (such as enzymes). In one example, metabolite pathway module 230 may generate alternative reaction selector 311 configured to display a selectable list (e.g., when hovered over) when two metabolites are connected by multiple metabolite reactions. UI module 232 then may update user interface 300 to display a selected metabolite reaction based on alternative reaction selector 311.

To further illustrate exploration of metabolite reaction data of reaction path 307, successor metabolite 326 is selected from successor metabolite list 323 of successor metabolite 324. As with FIG. 3C, FIG. 3D illustrates an update to the indicator corresponding to the continuation of reaction path 307 downward from successor metabolite 324. Similarly, as described above, the computing system 202 updates reaction path 307 of user interface 300 to show the successor metabolite 326 and its selectable precursor metabolite list 325 and successor metabolite list 327 and additionally any selected options from display options 318.

FIG. 3E illustrates automatically updating the user interface 300 when a precursor or successor metabolite is selected from an intermediate metabolite that resides between the metabolites on each end of the reaction path 307. For example, successor metabolite 328 associated with starting metabolite 304 is selected as shown in FIG. 3D. As a result, as illustrated in FIG. 3E, the computing system 202 updates reaction path 307 of user interface 300 to replace the existing portion of the reaction path 307 connected to the intermediate metabolite with successor metabolite 328 and its selectable successor metabolite list 329. In this example, successor metabolite 328 does not have any precursor metabolites to list in a precursor metabolite list.

In one example, when a portion of a reaction path (e.g., reaction path 307) is replaced, the prior reaction path is saved by the metabolite pathway module to a list of selectable previous paths, such as previous paths 330 of FIG. 3E. For example, if previous path 331 was selected from previous paths 330, the computing system 202 would update reaction path 307 of user interface 300 to revert back to the prior reaction path 307 that included successor metabolites 324 and 326 as shown in FIG. 3D. The computing system 202 is not limited with respect to the number of prior paths that may be stored and displayed in previous paths 330. This permits a user of computing system 202 to quickly navigate back and forth among previous paths without having to clear the user interface 300 and start over.

FIG. 3F illustrates how the reaction path 307 of user interface 300 updates when “show metabolite structures” is selected from display options 318. In one example, when selected, the computing system 202 updates the indicator corresponding to reaction path 307 to annotate the textual representations of the metabolites in reaction path 307 with their respective chemical compound structures as illustrated by chemical structure 332 corresponding to starting metabolite 304.

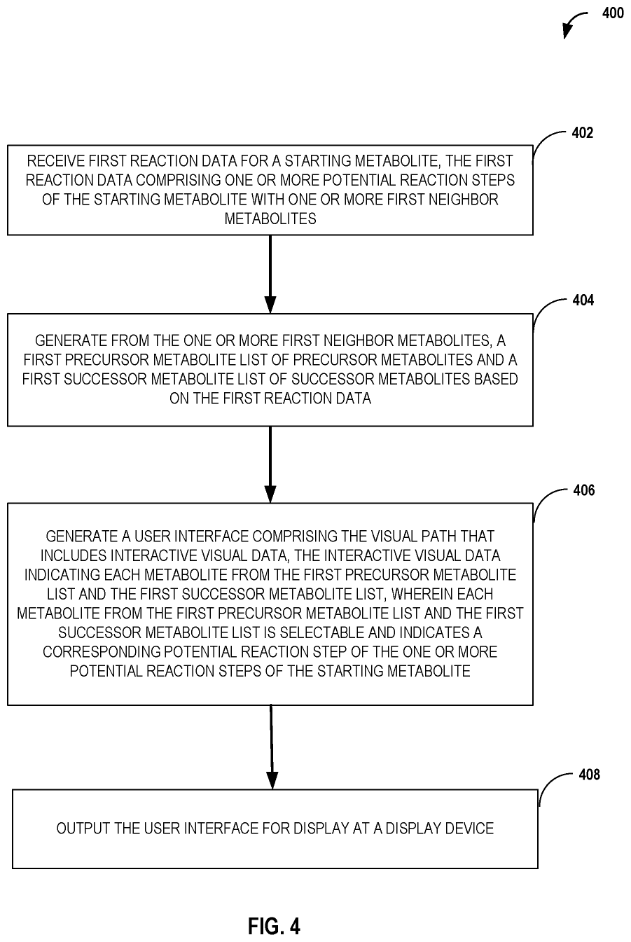

FIG. 4 is a flow diagram illustrating an example operation of a computer system for generating a visual path through a metabolic network in accordance with the techniques of the disclosure. FIG. 4 is described with respect to FIG. 2 for example purposes only. In the example of FIG. 4, computing system 202 generates a user interface (user interface 300) of a visual path through a metabolic network (e.g., reaction path 307) (400).

UI module 232 receives from metabolite database 214 reaction data 220 for a starting metabolite. The first reaction data includes one or more potential reaction steps from the starting metabolite to one or more neighbor metabolites (402). Metabolite pathway module 230 generates, from the one or more neighbor metabolites, a precursor metabolite list of precursor metabolites and a successor metabolite list of successor metabolites based on the reaction data 220 (404).

UI module 232 generates a user interface (e.g., user interface 300 of FIGS. 3A-3F) of the visual path (e.g., reaction path 307) through the metabolic network that includes interactive visual data, the interactive visual data indicating each metabolite from the precursor metabolite list and the successor metabolite list, wherein each metabolite from the precursor metabolite list and the successor metabolite list is selectable and indicates a corresponding potential reaction step of the one or more potential reaction steps of the starting metabolite (406). UI module 232 outputs the user interface for display at display device 224 of under interface devices 210 (408).

The computing devices described herein, such as host computing system 102, user device 122, and computing system 202, may include one or more processors or processing units to execute instructions, one or more memories to store information, one or more data input components to receive data input from a user of the computing device, a network interface communication circuit to establish a communication link to communicate with other computing devices external to the computing device, one or more sensors where an output from the sensors is used for sensing a specific triggering condition and then correspondingly generating one or more preprogrammed actions, a display screen to display at least some of the information stored in the one or more memories and other components. Note, portions of this design implemented in software are stored in the one or more memories and are executed by the one or more processors. The processing unit may have one or more processing cores, which couples to a system bus that couples various system components including the system memory. The system bus may be any of several types of bus structures selected from a memory bus, an interconnect fabric, a peripheral bus, and a local bus using any of a variety of bus architectures.

“Computing system” typically includes a variety of computing machine-readable media. Machine-readable media can be any available media that can be accessed by computing device and includes both volatile and nonvolatile media, and removable and non-removable media. By way of example, and not limitation, computing machine-readable media use includes storage of information, such as computer-readable instructions, data structures, other executable software, or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other tangible medium which can be used to store the desired information, and which can be accessed by the computing device. Transitory media such as wireless channels are not included in the machine-readable media. Machine-readable media typically embody computer readable instructions, data structures, and other executable software. In an example, the volatile memory can store portions of the operating system, application programs, other executable software, and program data.

The computing device can operate in a networked environment using logical connections to one or more remote computers/client devices, such as a remote computing system. The remote computing system can a personal computer, a mobile computing device, a server, a router, a network PC, a peer device, or other common network node, and typically includes many or all of the elements described above relative to the computing device. Such networking environments are commonplace in offices, enterprise-wide computer networks, intranets, and the Internet. A browser application and/or one or more local applications may be resident on the computing device and stored in the memory.

An application as described herein includes but is not limited to software applications, mobile applications, and programs that are part of an operating system application. Some portions of this description are presented in terms of algorithms and symbolic representations of operations on data bits within a computer memory. These algorithmic descriptions and representations are the means used by those skilled in the data processing arts to convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like. These algorithms can be written in a number of different software programming languages such as C, C++, HTTP, Java, or other similar languages. Also, an algorithm can be implemented with lines of code in software, configured logic gates in hardware, or a combination of both. In an embodiment, the logic consists of electronic circuits that follow the rules of Boolean Logic, software that contain patterns of instructions, or any combination of both. A module may be implemented in hardware electronic components, software components, and a combination of both.

Generally, applications include programs, routines, objects, widgets, plug-ins, and other similar structures that perform particular tasks or implement particular abstract data types. Those skilled in the art can implement the description and/or figures herein as computer-executable instructions, which can be embodied on any form of computing machine-readable media discussed herein.

Many functions performed by electronic hardware components can be duplicated by software emulation. Thus, a software program written to accomplish those same functions can emulate the functionality of the hardware components in input-output circuitry.

The foregoing system and embodiments thereof have been provided in sufficient detail, but it is not the intention of the applicant(s) for the disclosed system and embodiments provided herein to be limiting. Additional adaptations and/or modifications are possible, and, in broader aspects, these adaptations and/or modifications are also encompassed. Accordingly, departures may be made from the foregoing system and embodiments without departing from the spirit of the system.

The techniques described in this disclosure may be implemented, at least in part, in hardware, software, firmware or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components.

The techniques described in this disclosure may also be embodied or encoded in a computer-readable medium, such as a non-transitory computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

What is claimed is:

1. A method for generating a visual path through a metabolic network, the method comprising:
receiving, by a computing system, first reaction data for a starting metabolite, the first reaction data comprising one or more potential reaction steps of the starting metabolite with one or more first neighbor metabolites;
generating, by the computing system, from the one or more first neighbor metabolites, a first precursor metabolite list of precursor metabolites and a first successor metabolite list of successor metabolites based on the first reaction data;
generating, by the computing system, a user interface comprising the visual path that includes interactive visual data, the interactive visual data indicating each metabolite from the first precursor metabolite list and the first successor metabolite list, wherein each metabolite from the first precursor metabolite list and the first successor metabolite list is selectable and indicates a corresponding potential reaction step of the one or more potential reaction steps of the starting metabolite; and
outputting, by the computing system, the user interface for display at a display device.

2. The method of claim 1, further comprising.
receiving, by the computing system, a user input indicating selection of a precursor metabolite from the first precursor metabolite list to add a reaction step comprising the starting metabolite and the selected precursor metabolite;
receiving, by the computing system, in response to the user input, second reaction data for the selected precursor metabolite, the second reaction data comprising one or more potential reaction steps of the selected precursor metabolite with one or more second neighbor metabolites;
generating, from the one or more second neighbor metabolites, a second precursor metabolite list of precursor metabolites and a second successor metabolite list of successor metabolites based on the second reaction data;
modifying the user interface with updated interactive visual data, wherein the updated interactive visual data includes the reaction step comprising the starting metabolite and the selected precursor metabolite and an indicator indicating a first reaction from the selected precursor metabolite to the starting metabolite, wherein the updated interactive visual data indicates each metabolite from the second precursor metabolite list and the second successor metabolite list, and wherein each metabolite from the second precursor metabolite list and the second successor metabolite list is selectable and each indicates a corresponding potential reaction step of the one or more potential reaction steps of the selected precursor metabolite; and
outputting the modified user interface for display at the display device.

3. The method of claim 1, further comprising,
receiving, by the computing system, a user input indicating selection of a successor metabolite from the first successor metabolite list to add a reaction step comprising the starting metabolite and the selected successor metabolite;
receiving, by the computing system, in response to the user input, third reaction data for the selected successor metabolite, the third reaction data comprising one or more potential reaction steps of the selected successor metabolite with one or more third neighbor metabolites;
generating, from the one or more third neighbor metabolites, a third precursor metabolite list of precursor metabolites and a third successor metabolite list of successor metabolites based on the third reaction data;
modifying the user interface with updated interactive visual data, wherein the updated interactive visual data includes the reaction step comprising the starting metabolite and the selected successor metabolite, and an indicator indicating a first reaction from the selected successor metabolite to the starting metabolite, wherein the updated interactive visual data indicates each metabolite from the third precursor metabolite list and the third successor metabolite list, and wherein each precursor metabolite from the third precursor metabolite list and the third successor metabolite list is selectable and each indicates a corresponding potential reaction step of the one or more potential reaction steps of the selected successor metabolite; and
outputting the modified user interface for display at the display device.

4. The method of claim 3, wherein the updated interactive visual data includes the interactive visual data indicating each metabolite from the first precursor metabolite list and the first successor metabolite list of the starting metabolite, and wherein each metabolite from the first precursor metabolite list and the first successor metabolite list is selectable and indicates a corresponding potential reaction step of the one or more potential reaction steps of the starting metabolite.

5. The method of claim 3, the method further comprising:
storing previous reaction path data comprising the reaction step corresponding to one or more added precursor and successor metabolites to the starting metabolite, and wherein the updated interactive visual data comprises previous path data selectable within the modified user interface.

6. The method of claim 1, wherein the user interface comprises a plurality of pathway display options for display on the user interface, wherein each of the pathway display options is selectable, the method further comprising;
receiving, by the computing system, a user input indicating a selection of one or more pathway display options;
updating the interactive visual data of the user interface with the selected one or more pathway display options; and
outputting the updated user interface for display at the display device.

7. The method of claim 6, wherein the user input is an indication of a selection to include a chemical structure for each metabolite displayed on the user interface, and wherein the updating the interactive visual data of the user interface further comprises:
updating the interactive visual data of the user interface with the chemical structure for each metabolite displayed on the user interface.

8. The method of claim 6, wherein the user input is an indication of a selection of a pathway display option to display text corresponding to the reaction data, and wherein the updating the interactive visual data of the user interface further comprises:
updating the user interface to include the text corresponding to the reaction data.

9. The method of claim 6, wherein the user input to display the text corresponding to the reaction data is a hover selection input, and wherein the updating the interactive visual data of the user interface further comprises:
updating, in response to the hover selection input, the user interface to include a pop-up element comprising the text corresponding to the reaction data.

10. The method of claim 9, wherein the user interface comprising the visual path includes interactive visual data that indicates a selectable list of a metabolite reactions when the reaction path includes two metabolites connected by multiple reactions.

11. A metabolic network exploration system comprising:
a storage device; and
processing circuitry having access to the storage device and configured to:
receive first reaction data for a starting metabolite, the first reaction data comprising one or more potential reaction steps of the starting metabolite with one or more first neighbor metabolites;
generate from the one or more first neighbor metabolites, a first precursor metabolite list of precursor metabolites and a first successor metabolite list of successor metabolites based on the first reaction data;
generate a user interface comprising the visual path that includes interactive visual data, the interactive visual data indicating each metabolite from the first precursor metabolite list and the first successor metabolite list, wherein each metabolite from the first precursor metabolite list and the first successor metabolite list is selectable and indicates a corresponding potential reaction step of the one or more potential reaction steps of the starting metabolite; and
output the user interface for display at a display device.

12. The system of claim 11, wherein the processing circuitry is configured to:
receive, by the computing system, a user input indicating selection of a precursor metabolite from the first precursor metabolite list to add a reaction step comprising the starting metabolite and the selected precursor metabolite;
receive, by the computing system, in response to the user input, second reaction data for the selected precursor metabolite, the second reaction data comprising one or more potential reaction steps of the selected precursor metabolite with one or more second neighbor metabolites;
generate, from the one or more second neighbor metabolites, a second precursor metabolite list of precursor metabolites and a second successor metabolite list of successor metabolites based on the second reaction data;
modify the user interface with updated interactive visual data, wherein the updated interactive visual data includes the reaction step comprising the starting metabolite and the selected precursor metabolite and an indicator indicating a first reaction from the selected precursor metabolite to the starting metabolite, wherein the updated interactive visual data indicates each metabolite from the second precursor metabolite list and the second successor metabolite list, and wherein each metabolite from the second precursor metabolite list and the second successor metabolite list is selectable and each indicates a corresponding potential reaction step of the one or more potential reaction steps of the selected precursor metabolite; and
output the modified user interface for display at the display device.

13. The system of claim 11, wherein the processing circuitry is configured to:
receive, by the computing system, a user input indicating selection of a successor metabolite from the first successor metabolite list to add a reaction step comprising the starting metabolite and the selected successor metabolite;
receive, by the computing system, in response to the user input, third reaction data for the selected successor metabolite, the third reaction data comprising one or more potential reaction steps of the selected successor metabolite with one or more third neighbor metabolites;
generate, from the one or more third neighbor metabolites, a third precursor metabolite list of precursor metabolites and a third successor metabolite list of successor metabolites based on the third reaction data;
modify the user interface with updated interactive visual data, wherein the updated interactive visual data includes the reaction step comprising the starting metabolite and the selected successor metabolite, and an indicator indicating a first reaction from the selected successor metabolite to the starting metabolite, wherein the updated interactive visual data indicates each metabolite from the third precursor metabolite list and the third successor metabolite list, and wherein each precursor metabolite from the third precursor metabolite list and the third successor metabolite list is selectable and each indicates a corresponding potential reaction step of the one or more potential reaction steps of the selected successor metabolite; and
output the modified user interface for display at the display device.

14. The system of claim 13, wherein the updated interactive visual data includes the interactive visual data indicating each metabolite from the first precursor metabolite list and the first successor metabolite list of the starting metabolite, and wherein each metabolite from the first precursor metabolite list and the first successor metabolite list is selectable and indicates a corresponding potential reaction step of the one or more potential reaction steps of the starting metabolite.

15. The system of claim 13, wherein the processing circuitry is configured to:
store previous reaction path data including the reaction step corresponding to one or more added precursor and successor metabolites to the starting metabolite, and wherein the updated interactive visual data comprises previous path data selectable within the modified user interface.

16. The system of claim 11, wherein the user interface comprises a plurality of pathway display options for display on the user interface, wherein each of the pathway display options is selectable, and wherein the processing circuitry is configured to;
receive, by the computing system, a user input indicating a selection of one or more pathway display options;
update the interactive visual data of the user interface with the selected one or more pathway display options; and
output the updated user interface for display at the display device.

17. The system of claim 16, wherein the user input is an indication of a selection to include a chemical structure for each metabolite displayed on the user interface, and wherein to update the interactive visual data of the user interface the processing circuitry is configured to update the interactive visual data of the user interface with the chemical structure for each metabolite displayed on the user interface.

18. The system of claim 16, wherein the user input is an indication of a selection of a pathway display option to display text corresponding to the reaction data, and wherein to update the interactive visual data of the user interface the processing circuitry is configured to update the user interface to include the text corresponding to the reaction data.

19. The system of claim 16, wherein the user input to display the text corresponding to the reaction data is a hover selection input, and wherein to update the interactive visual data of the user interface the processing circuitry is configured to update, in response to the hover selection input, the user interface to include a pop-up element comprising the text corresponding to the reaction data.

20. A non-transitory, computer-readable medium comprising instructions for causing one or more programmable processors to:
receive first reaction data for a starting metabolite, the first reaction data comprising one or more potential reaction steps of the starting metabolite with one or more first neighbor metabolites;
generate from the one or more first neighbor metabolites, a first precursor metabolite list of precursor metabolites and a first successor metabolite list of successor metabolites based on the first reaction data;
generate a user interface comprising the visual path that includes interactive visual data, the interactive visual data indicating each metabolite from the first precursor metabolite list and the first successor metabolite list, wherein each metabolite from the first precursor metabolite list and the first successor metabolite list is selectable and indicates a corresponding potential reaction step of the one or more potential reaction steps of the starting metabolite; and
output the user interface for display at a display device.
